(19) United States
(12) Patent Application Publication
(10) Pub. No.: US 2018/0202976 A1
(43) Pub. Date: Jul. 19, 2018

(54) SENSOR CIRCUIT AND SENSING METHOD

(71) Applicant: TAIYO YUDEN CO., LTD., Tokyo (JP)

(72) Inventor: Tetsuo SAJI, Tokyo (JP)

(73) Assignee: TAIYO YUDEN CO., LTD., Tokyo (JP)

(21) Appl. No.: 15/839,401

(22) Filed: Dec. 12, 2017

(30) Foreign Application Priority Data
Jan. 17, 2017 (JP) 2017-006151

(51) Int. Cl. G01N 29/02; G01N 29/036; G01N 29/30; G01R 23/02; G01N 29/24; G01N 33/00

(52) U.S. Cl. CPC G01N 29/022; G01N 29/036; G01N 29/30; G01N 2291/012; G01N 29/2437; G01N 33/0031; G01R 23/02

(57) ABSTRACT

A sensor circuit includes: a resonator of which a resonant frequency and/or an antiresonant frequency changes as a mass of a sensitive part of the resonator changes; an amplifier outputting an oscillation signal having a frequency corresponding to the resonant frequency or the antiresonant frequency; a phase shift circuit changing a phase difference between a first signal and a second signal branched from the oscillation signal in accordance with a change in frequency of the oscillation signal; and a mixer outputting a signal corresponding to a change in the resonant frequency or the antiresonant frequency of the resonator by mixing the first signal and the second signal between which the phase difference has been changed by the phase shift circuit.

### CROSS-REFERENCE TO RELATED APPLICATION

This application is based upon and claims the benefit of priority of the prior Japanese Patent Application No. 2017-006151, filed on Jan. 17, 2017, the entire contents of which are incorporated herein by reference.

### FIELD

A certain aspect of the present invention relates to a sensor circuit and a sensing method.

### BACKGROUND

There have been known environmental sensors that detect a physical quantity such as, for example, the concentration of specific atoms or specific molecules in a gas or a liquid, temperature, or humidity by detecting a change in mass of a sensitive membrane. There has been known a sensor circuit that has an acoustic wave resonator having a sensitive membrane (a surface detecting a substance) as a phase shifter and detects a substance based on the phase shift amount of a reference oscillation signal as disclosed in, for example, U.S. Pat. No. 5,932,953 (hereinafter, referred to as Patent Document 1). There have been also known sensor circuits that detect a substance based on a difference in resonant frequency between an acoustic wave resonator having a sensitive membrane (a reactive film or a chemical interactive film detecting a substance) and a reference acoustic wave resonator as disclosed in, for example, Japanese Patent Application Publication Nos. 2004-226405 and 2008-544259 (hereinafter, referred to as Patent Documents 2 and 3).

### SUMMARY OF THE INVENTION

According to the first aspect of the present invention, there is provided a sensor circuit including: a resonator of which a resonant frequency and/or an antiresonant frequency changes as a mass of a sensitive part of the resonator changes; an amplifier outputting an oscillation signal having a frequency corresponding to the resonant frequency or the antiresonant frequency; a phase shift circuit changing a phase difference between a first signal and a second signal branched from the oscillation signal in accordance with a change in frequency of the oscillation signal; and a mixer outputting a signal corresponding to a change in the resonant frequency or the antiresonant frequency of the resonator by mixing the first signal and the second signal between which the phase difference has been changed by the phase shift circuit.

According to the second aspect of the present invention, there is provided a sensing method including: outputting an oscillation signal having a frequency corresponding to a resonant frequency or an antiresonant frequency of a resonator, the resonant frequency or the antiresonant frequency changing as a mass of a sensitive part of the resonator changes; changing a phase difference between a first signal and a second signal branched from the oscillation signal in accordance with a change in frequency of the oscillation signal; and outputting a signal corresponding to a change in the resonant frequency or the antiresonant frequency of the resonator by mixing the first signal and the second signal between which the phase difference has been changed.

### BRIEF DESCRIPTION OF THE DRAWINGS

FIG. 1 is a circuit diagram of a sensor circuit in accordance with a first embodiment;

FIG. 2 is a graph of voltage versus time for each signal in the first embodiment;

FIG. 3 is a graph of the voltage of a signal S5 versus the phase difference between signals S2 and S3 in the first embodiment;

FIG. 4 illustrates the phase shift amounts of phase shifters with respect to frequency in the first embodiment;

FIG. 5 is a graph of an S3−S2 phase difference and the voltage of the signal S5 versus a frequency shift of an oscillation signal in the first embodiment;

FIG. 6A is a plan view of an example of a resonator in the first embodiment, and FIG. 6B is a cross-sectional view taken along line A-A in FIG. 6A;

FIG. 7 is a circuit diagram of an example of an oscillation circuit in the first embodiment;

FIG. 8 presents the transmission characteristic of the resonator and the phase shift amount of the phase shifter in the first embodiment;

FIG. 9 is a circuit diagram of another example of the oscillation circuit in the first embodiment;

FIG. 10A through FIG. 10C are circuit diagrams of examples of the phase shifter in the first embodiment;

FIG. 11A and FIG. 11B illustrate the phase shift amount with respect to frequency in the phase shifters illustrated in FIG. 10A and FIG. 10B, respectively;

FIG. 12 presents the transmission characteristic and the phase shift amount of the phase shifter illustrated in FIG. 10B;

FIG. 13A is a circuit diagram of the phase shifter in the first embodiment, and FIG. 13B illustrates the phase shift amount of the phase shifter with respect to frequency;

FIG. 14 is a circuit diagram of a sensor circuit in accordance with a second embodiment;

FIG. 15 is a flowchart of a sensing method in the second embodiment;

FIG. 16A and FIG. 16B illustrate other examples of the acoustic wave resonator of the resonator in the first and second embodiments;

FIG. 17A and FIG. 17B illustrate yet other examples of the acoustic wave resonator of the resonator in the first and second embodiments;

FIG. 18 is a plan view of examples of the acoustic wave resonators of the resonator and the phase shifter in the first and second embodiments;

FIG. 19A is a cross-sectional view taken along line A-A in FIG. 18, and FIG. 19B is a cross-sectional view taken along line B-B in FIG. 18;

FIG. 20A is another cross-sectional view taken along line A-A in FIG. 18, and FIG. 20B is another cross-sectional view taken along line B-B in FIG. 18;

FIG. 21 is a plan view of an additional film in the first and second embodiments; and

FIG. 22A and FIG. 22B are cross-sectional views of the sensor circuit in the first and second embodiments.

### DETAILED DESCRIPTION

In Patent Document 1, the acoustic wave resonator having a sensitive membrane has a small Q-value. Thus, the phase shift amount with respect to the mass change of the sensitive membrane is small, and the detection sensitivity is thus low. In Patent Documents 2 and 3, two oscillators each including an acoustic wave resonator need to be used, leading to increase in circuit size.

Hereinafter, with reference to the accompanying drawings, embodiments will be described.

### First Embodiment

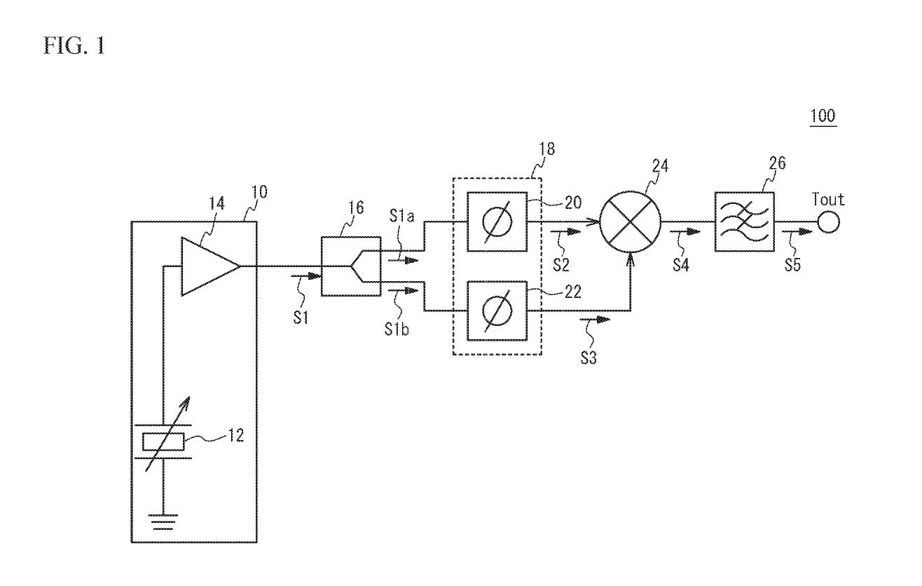

FIG. 1 is a circuit diagram of a sensor circuit in accordance with a first embodiment. A sensor circuit 100 includes an oscillation circuit 10, a branch circuit 16, a phase shift circuit 18, a mixer 24, and a low-pass filter (LPF) 26.

The oscillation circuit 10 has a resonator 12 and an amplifier 14. The resonator 12 changes its resonant frequency and/or antiresonant frequency in accordance with a change in mass of its sensitive part. The sensitive part is a part of which the mass changes in accordance with an environmental change. For example, when specific atoms or specific molecules in a gas or a liquid adsorb to the sensitive part, the mass of the sensitive part increases. When the humidity of the atmosphere increases, water adsorbs to the sensitive part, increasing the mass of the sensitive part. A change in temperature changes the mass of the sensitive part. The irradiation of the sensitive part with light such as ultraviolet light changes the mass of the sensitive part. The amplifier 14 functions as an oscillator, and outputs an oscillation signal S1 having a frequency corresponding to the resonant frequency or the antiresonant frequency of the resonator.

The branch circuit 16 is, for example, a power splitter, and branches the oscillation signal S1 into signals S1a and S1b that have substantially identical frequencies, substantially identical phases, and substantially identical powers. The phase shift circuit 18 has phase shifters 20 and 22. The phase shifter 20 shifts the phase of the signal S1a and outputs a signal S2. The phase shifter 22 shifts the phase of the signal S1b and outputs a signal S3. The phase difference between the signals S2 and S3 varies according to the frequency of the oscillation signal S1. For example, the phase shifter 20 changes the shift amount of the phase in accordance with a change in frequency of the signal S1a. In the phase shifter 22, the phase shift amount remains nearly unchanged irrespective of the frequency of the signal S1a.

The mixer 24 is a multiplier, and outputs a signal S4 resulting from mixing (multiplication) of the signals S2 and S3. The LPF 26 has a cutoff frequency lower than the frequency of the oscillation signal S1, filters the signal S4, and outputs a signal S5 with a frequency component lower than the frequency of the oscillation signal S1 to an output terminal Tout.

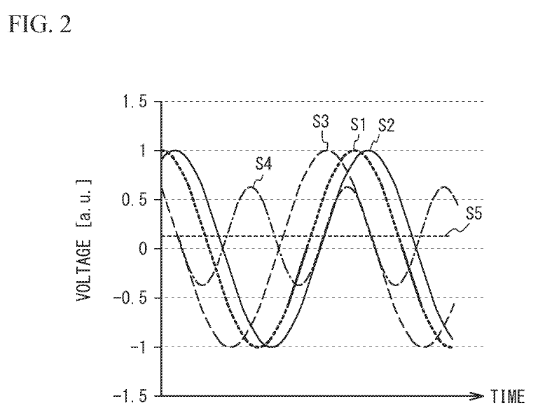

FIG. 2 is a graph of voltage versus time for each signal in the first embodiment. Time and voltage are presented in arbitrary units (a.u.). As illustrated in FIG. 2, it is assumed that the oscillation signal S1 is a sine wave signal. The oscillation signal S1 is expressed by the following formula 1. A0 represents amplitude.

S1=A0·cos(ωt) (1)

The phase shifter 20 makes the phase of the signal S2 lag behind the phase of the oscillation signal S1. The phase shifter 22 makes the phase of the signal S3 ahead of the phase of the oscillation signal. The signals S2 and S3 are respectively expressed by the following formulas 2 and 3. A1 and A2 represent amplitudes. As presented in the formulas 2 and 3, the frequencies of the signals S2 and S3 are identical to the frequency of the oscillation signal S1, and the phase of the signals S2 and S3 differ from each other.

S2=A1·cos(ωt+θ1) (2)

S3=A2·cos(ωt+θ2) (3)

The mixer 24 multiplies the signal S2 by the signal S3. The signal S4 is expressed by the following formula 4. The signal S4 mainly has a frequency component approximately twice the frequency of the oscillation signal and a frequency component corresponding to the phase difference θ1-θ2 between the signals S2 and S3.

$$\begin{matrix}S 4 = A 1 \cdot cos ( ω t + θ 1 ) \times A 2 \cdot cos ( ω t + θ 2 ) \\ = 0.5 \cdot A 1 \cdot A 2 \cdot \{ cos ( θ 1 - θ 2 ) + A 2 \cdot cos ( 2 ω t + θ 1 + θ 2 ) \}\end{matrix} \qquad ( 4 )$$

The LPF 26 removes the frequency component twice the frequency of the oscillation signal S1 from the signal S4. The signal S5 is expressed by the following formula 5. As presented in the formula 5, the signal S5 has a frequency component corresponding to the phase difference θ1-θ2. The frequency corresponding to the phase difference θ1-θ2 is sufficiently smaller than the frequency of the oscillation signal S1, and thus, is considered to be a direct current component with respect to the frequency of the oscillation signal S1.

S5=0.5·A1·A2·cos(θ1−θ2) (5)

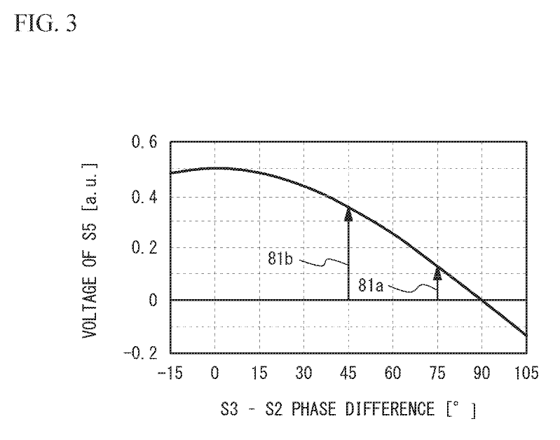

FIG. 3 is a graph of the voltage of the signal S5 versus the phase difference between the signals S2 and S3 in the first embodiment. The voltage is presented in an arbitrary unit, and the arbitrary unit is, for example, V. As illustrated in FIG. 3, when the phase difference is 90°, the voltage of the signal S5 is 0. When the phase difference becomes smaller than 90°, the voltage of the signal S5 increases. When the phase difference is 0°, the voltage of the signal S5 is 0.5. As described above, as the phase difference between the signals S2 and S3 changes, the voltage of the signal S5 changes. When the phase difference of S3−S2 is 90°, the slope of the voltage of the signal S5 with respect to the S3−S2 phase difference has the maximum value. Accordingly, in the viewpoint of detection sensitivity, the phase difference of S3−S2 is preferably around 90°.

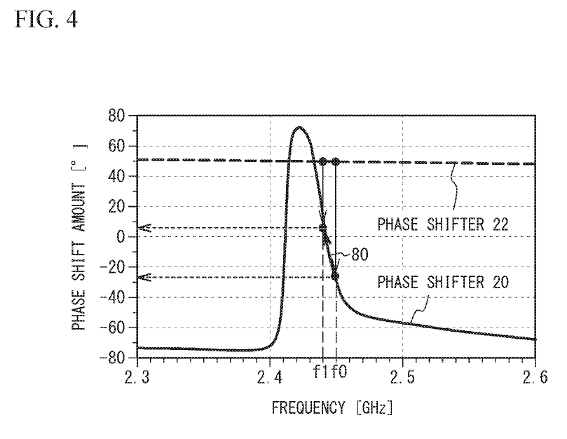

FIG. 4 illustrates the phase shift amounts of the phase shifters with respect to frequency in the first embodiment. The solid line indicates the phase shift amount of the phase shifter 20, and the dashed line indicates the phase shift amount of the phase shifter 22. As illustrated in FIG. 4, the phase shifter 20 mainly delays the phase (the phase shift amount is negative). In a range from 2.4 GHz to 2.45 GHz, the phase shift amount of the phase shifter 20 has a peak. Around the peak of the phase shift amount, the phase shift amount is positive (the phase advances). The phase shifter 22 advances the phase (the phase shift amount is positive). The phase shift amount of the phase shifter 22 hardly depends on frequency.

Between 2.43 GHz and 2.45 GHz, the phase shift amount of the phase shifter 20 substantially linearly changes rapidly with respect to frequency. Assumed is a case where the frequency of the oscillation signal S1 lowers when the sensor circuit starts sensing operation. In this case, it is assumed that a reference frequency f0 in an initial state prior to the sensing operation of the sensor circuit is around the higher frequency end of the frequency range in which the phase shift amount substantially linearly changes rapidly. Additionally, it is assumed that the S3−S2 phase difference at the reference frequency f0 is around 90° as illustrated in FIG. 3. Under these assumptions, the reference frequency f0 and the phase shift amount at the reference frequency f0 are assumed as follows in the example of FIG. 4.

Reference frequency f0: 2.45 GHz

Phase shift amount of the phase shifter 20: −25°

Phase shift amount of the phase shifter 22: +50°

Phase difference of the signals S3−S2: +75°

It is assumed that the mass of the sensitive part increases and the resonant frequency decreases when the sensor circuit starts sensing operation. For example, it is assumed that the frequency f1 of the oscillation signal S1 and the phase shift amount at the frequency f1 change as indicated by an arrow 80.

Frequency f1: 2.44 GHz

Phase shift amount of the phase shifter 20: +5°

Phase shift amount of the phase shifter 22: +50°

Phase difference of the signals S3−S2: +45°

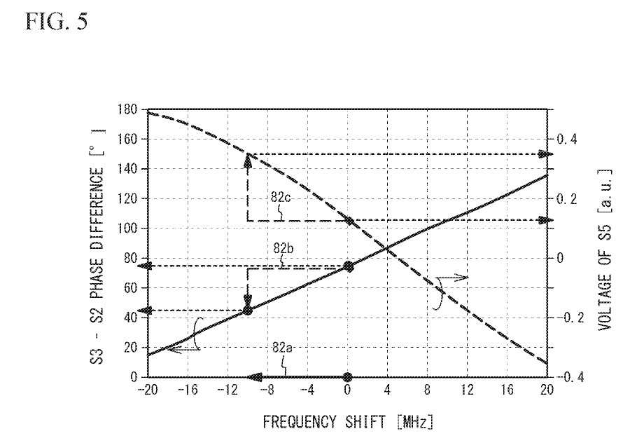

FIG. 5 is a graph of the S3−S2 phase difference and the voltage of the signal S5 versus the frequency shift of the oscillation signal in the first embodiment. The solid line indicates the phase difference, and the dashed line indicates the voltage of the signal S5. The frequency shift is a frequency shift from the reference frequency f0 at the time of sensing operation. In FIG. 4, the frequency shift is 0 MHz at the reference frequency f0 (2.45 GHz), and the frequency shift is −10 MHz at the frequency f1 (2.44 GHz). When the frequency shift is 0 MHz, the signal S3−S2 phase difference is 75° as illustrated in FIG. 4. In this case, as indicated by an arrow 81a in FIG. 3, the voltage of the signal S5 is 0.13. When the frequency shift is −10 MHz, the signal S3−S2 phase difference is 45° as illustrated in FIG. 4. As indicated by an arrow 81b in FIG. 3, the voltage of the signal S5 is 0.37. Thus, when the frequency shift changes from 0 MHz to −10 MHz as indicated by an arrow 82a in FIG. 5, the S3−S2 phase difference changes from 75° to 45° as indicated by an arrow 82b and the voltage of the signal S5 changes from 0.13 to 0.37 as indicated by an arrow 82c.

As described above, the resonant frequency of the resonator 12 is set at the reference frequency f0. As the mass of the sensitive part increases, the resonant frequency of the resonator 12 decreases to the frequency f1. Accordingly, the frequency of the oscillation signal S1 changes from f0 to f1. As illustrated in FIG. 4, the phase difference between the signals S3 and S2 decreases. As illustrated in FIG. 5, the shift from the reference frequency f0 changes the voltage of the signal S5. Accordingly, the mass change of the sensitive part is converted into the change in voltage of the signal S5.

The relation between the voltage of the signal S5 and the physical quantity to be detected (for example, the concentration of specific molecules in a gas or a liquid, temperature, humidity, or an amount of ultraviolet light) is obtained in advance. Use of the relation obtained in advance allows the physical quantity to be detected based on the voltage of the signal S5.

In the first embodiment, the resonant frequency and/or the antiresonant frequency of the resonator 12 changes as the mass of the sensitive part changes. The amplifier 14 functioning as an oscillator outputs the oscillation signal S1 having a frequency corresponding to the resonant frequency or the antiresonant frequency. The phase shift circuit 18 changes the phase difference between the signals S1a (a first signal) and S1b (a second signal) branched from the oscillation signal S1 in accordance with a change in frequency of the oscillation signal S1. The mixer 24 outputs a signal corresponding to a change in the resonant frequency or the antiresonant frequency of the resonator 12 by mixing the signals S2 and S3 between which the phase difference has been changed by the phase shift circuit 18.

Since the number of oscillators is one, the sensor circuit is reduced in size compared with Patent Documents 2 and 3. Additionally, measurement errors such as fluctuations between oscillation frequencies due to the provision of a plurality of oscillators are reduced. Additionally, the phase shifter 20 has no sensitive part. Accordingly, the phase shifter 20 has a high Q-value, and thus, the detection sensitivity to the frequency shift can be made to be high.

As illustrated in FIG. 4, the phase shifter 20 (a first phase shifter) changes the phase of the signal S1a by a first phase shift amount. The phase shifter 22 (a second phase shifter) changes the phase of the signal S1b by a second phase shift amount. The amount of change in the second phase shift amount with respect to a change in frequency of the signal S1a differs from the amount of change in the first phase shift amount with respect to a change in frequency of the signal S1a. This configuration allows the frequency shift associated with the mass change of the sensitive part to be detected as illustrated in FIG. 5.

To increase the frequency dependence of the phase difference between the signals S3 and S2, the slope of the second phase shift amount of the phase shifter 22 with respect to frequency is preferably close to 0. Furthermore, the slope of the phase shift amount of the phase shifter 20 with respect to frequency is preferably opposite in sign to the slope of the phase shift amount of the phase shifter 22 with respect to frequency.

Furthermore, the LPF 26 having a cutoff frequency lower than the frequency of the oscillation signal S1 is preferably coupled to the output terminal of the mixer 24. This configuration enables to output the frequency shift as a direct current signal. The cutoff frequency of the LPF 26 is more preferably less than the half of the frequency of the oscillation signal S1.

### Example of the Resonator

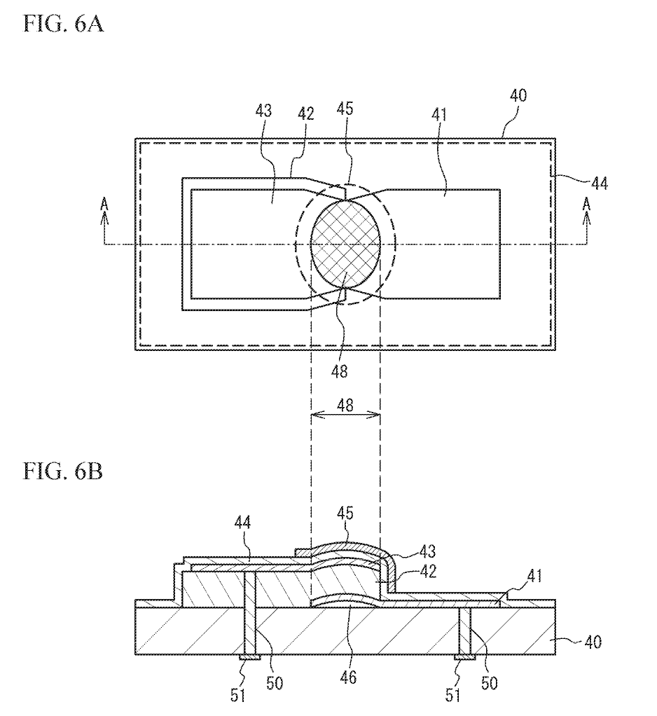

A case where a piezoelectric thin film resonator is used as the resonator will be described. FIG. 6A is a plan view of an example of the resonator in the first embodiment, and FIG. 6B is a cross-sectional view taken along line A-A in FIG. 6A. As illustrated in FIG. 6A and FIG. 6B, a piezoelectric film 42 is located on a substrate 40. A lower electrode 41 and an upper electrode 43 are located so as to sandwich the piezoelectric film 42. An air gap 46 is formed between the lower electrode 41 and the substrate 40. A resonance region 48 is a region in which the lower electrode 41 and the upper electrode 43 face each other across the piezoelectric film 42. In the resonance region 48, the lower electrode 41 and the upper electrode 43 excite the acoustic wave in the thickness extension mode inside the piezoelectric film 42. A protective film 44 is located on the substrate 40 so as to cover the lower electrode 41, the piezoelectric film 42, and the upper electrode 43. A sensitive membrane 45 is located on the protective film 44. In plan view, the sensitive membrane 45 includes the resonance region 48. Electrodes 51 are located on the lower surface of the substrate 40. Through electrodes 50 penetrating through the substrate 40 and the piezoelectric film 42 are provided. The through electrodes 50 connect the lower electrode 41 and the upper electrode 43 to the electrodes 51.

When gaseous molecules or liquid molecules adsorb to the sensitive membrane 45, the mass of the sensitive membrane 45 increases. When temperature or humidity changes, the mass of the sensitive membrane 45 changes. As the mass of the sensitive membrane 45 within the resonance region 48 increases, the resonant frequency and the antiresonant frequency of the piezoelectric thin film resonator decreases.

The substrate 40 is, for example, a sapphire substrate, an alumina substrate, a spinel substrate, or a silicon substrate. The lower electrode 41 and the upper electrode 43 are formed of a metal film such as, for example, a ruthenium (Ru) film. The piezoelectric film 42 is formed of, for example, an aluminum nitride (AlN) film, a zinc oxide (ZnO) film, or a crystal layer. The protective film 44 is an insulating film such as, for example, a silicon oxide film or a silicon nitride film. The through electrode 50 and the electrode 51 are formed of a metal layer such as, for example, a gold (Au) layer or a copper (Cu) layer.

The sensitive membrane 45 corresponds to the sensitive part. The sensitive membrane 45 may be made of an organic polymer film, an organic low molecular film, or an inorganic film. The sensitive membrane 45 may be formed by dissolving the material of the sensitive membrane into a solvent and then coating the resultant solvent, evaporation, sputtering, or chemical vapor deposition (CVD).

The organic polymeric material may be, for example, a homopolymer made of a single structure such as polystyrene, polymethylmethacrylate, 6-nylon, cellulose acetate, poly-9,9-dioctyl fluorene, polyvinyl alcohol, polyvinyl carbazole, polyethylene oxide, polyvinyl chloride, poly-p-phenylene ether sulfone, poly-1-butene, polybutadiene, polyphenyl methyl silane, polycaprolactone, poly bis phenoxyphosphazene, or polypropylene, a copolymer of different homopolymers, or a blend polymer that is a mixture of a homopolymer and a copolymer.

For example, the organic low molecular material may be tris(8-quinolinolato) aluminum (Alq3), naphthyl diamine (α-NPD), 2,9-dimethyl-4,7-diphenyl-1,10-phenanthroline (BCP), 4,4′-N,N′-dicarbazole-biphenyl (CBP), copper phthalocyanine, fullerene, pentacene, anthracene, thiophene, Ir(ppy(2-phenylpyridinato))₃, triazinethiol derivative, dioctyl fluorene derivative, tetracontane, or parylene.

For example, the inorganic material may be alumina, titania, vanadium pentoxide, tungsten oxide, lithium fluoride, magnesium fluoride, aluminum, gold, silver, tin, indium tin oxide (ITO), carbon nanotube, sodium chloride, or magnesium chloride.

Instead of the air gap 46, an acoustic mirror, which reflects the acoustic wave propagating through the piezoelectric film 42 in the longitudinal direction, may be used. The planar shape of the resonance region 48 may be, instead of an elliptical shape, a polygonal shape such as a quadrangle shape or a pentagonal shape.

### Example of the Oscillation Circuit

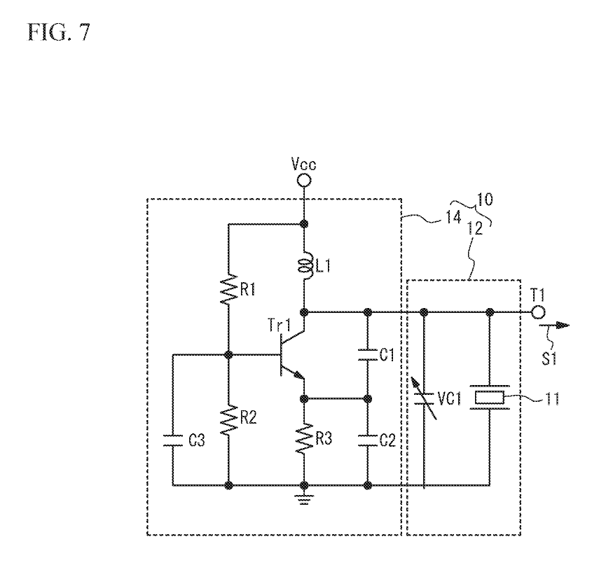

FIG. 7 is a circuit diagram of an example of the oscillation circuit in the first embodiment. As illustrated in FIG. 7, the oscillation circuit 10 includes the resonator 12 and the amplifier 14. The resonator 12 has an acoustic wave resonator 11 and a variable capacitor VC1. The acoustic wave resonator 11 is, for example, the piezoelectric thin film resonator illustrated in FIG. 6A and FIG. 6B. The acoustic wave resonator 11 and the variable capacitor VC1 are connected in parallel between an output terminal T1 and a ground.

The amplifier 14 has a transistor Tr1, resistors R1 through R3, capacitors C1 through C3, and an inductor L1. The emitter of the transistor Tr1 is coupled to a ground via the resistor R3 and the capacitor C2 connected in parallel to each other. The base of the transistor Tr1 is coupled to the ground via the resistor R2 and the capacitor C3 connected in parallel to each other, and is coupled to a power source terminal Vcc via the resistor R1. The collector of the transistor Tr1 is coupled to the power source terminal via the inductor L1, to the emitter via the capacitor C1, and to the output terminal T1.

The resistors R1 and R2 define the bias voltage supplied to each terminal of the transistor Tr1. The inductor L1 inhibits high-frequency signals from leaking to the power source terminal Vcc. The capacitors C1 through C3 are positively fed back the output of the collector to the base.

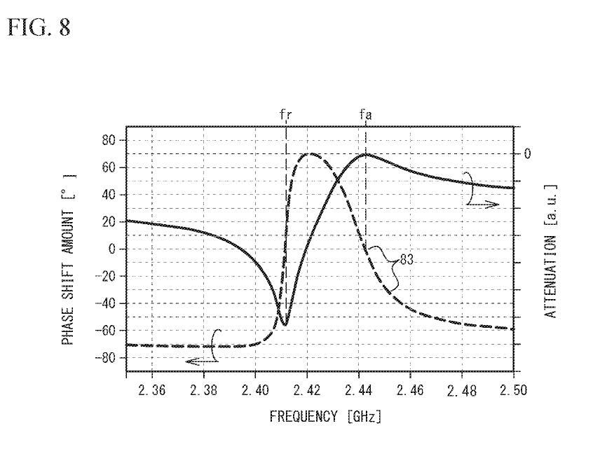

FIG. 8 is a graph of the transmission characteristic of the resonator and the phase shift amount of the phase shifter in the first embodiment. The solid line indicates an exemplary transmission characteristic of the resonator 12 (the transmission characteristic from the collector of the transistor Tr1 to the output terminal T1). The resonant frequency fr and the antiresonant frequency fa respectively correspond to the resonant frequency and the antiresonant frequency of the resonator 12. The dashed line indicates an exemplary phase shift amount of the phase shifter 20. As presented in FIG. 8, the attenuation of the resonator 12 is large at the resonant frequency fr, and the attenuation is small at the antiresonant frequency fa. Accordingly, the oscillation circuit 10 outputs the oscillation signal S1 with a frequency corresponding to the antiresonant frequency fa. In the resonator 12, as the capacitance of the variable capacitor VC1 is changed, the antiresonant frequency fa changes. Accordingly, the frequency of the oscillation signal S1 can be adjusted by adjusting the variable capacitor VC1.

The antiresonant frequency fa of the resonator 12 is adjusted to be at a frequency around the higher frequency end of the frequency range within which the phase shift amount of the phase shifter 20 greatly varies (a range 83: for example, a range in which the phase shift amount is from 0° to −45°). This adjustment enables to detect an increase in mass of the sensitive membrane of the acoustic wave resonator 11 with high sensitivity.

In the piezoelectric thin film resonator illustrated in FIG. 6A and FIG. 6B, as the mass of the sensitive membrane 45 changes, the antiresonant frequency changes more than the resonant frequency. Thus, to improve the detection sensitivity, the oscillation circuit 10 preferably oscillates at the antiresonant frequency of the resonator 12. As described above, when the phase shifter 20 is composed of an acoustic wave resonator, the range 83 corresponds to the range around of the antiresonant frequency of the acoustic wave resonator. Therefore, when acoustic wave resonators having similar structures are used for the resonator 12 and the phase shifter 20, the frequency temperature characteristic of the reference frequency f0 is made to be substantially identical to the frequency temperature characteristic of the phase shift amount of the phase shifter 20 by setting the antiresonant frequency fa of the resonator 12 at the oscillation frequency. Accordingly, the temperature characteristic of the sensor circuit is improved.

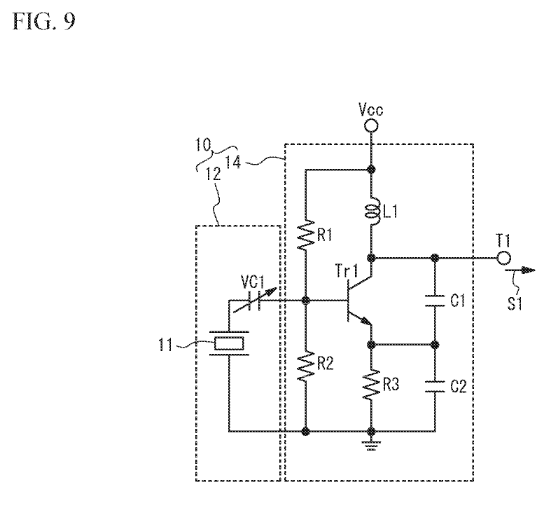

FIG. 9 is a circuit diagram of another example of the oscillation circuit in the first embodiment. The structure illustrated in FIG. 9 differs from that in FIG. 7 in that the resonator 12 is connected between the base of the transistor Tr1 and the ground. The acoustic wave resonator 11 and the variable capacitor VC1 are connected in series. Other structures are the same as those of FIG. 7, and the description thereof is thus omitted.

In the example of FIG. 9, the base of the transistor Tr1 is grounded with low impedance at the resonant frequency of the resonator 12. Therefore, the frequency of the oscillation signal S1 of the oscillation circuit 10 is the resonant frequency fr of the resonator 12. The resonant frequency of the resonator 12 can be adjusted by adjusting the variable capacitor VC1. For example, the resonant frequency fr is configured to be within the range 83 in FIG. 8. The resonant frequency fr of the resonator 12 greatly varies according to the capacitance of the variable capacitor VC1. Thus, this structure is suitable for drastically adjusting the frequency of the oscillation signal S1.

As described above, the use of the acoustic wave resonator 11 (a second acoustic wave resonator) for the resonator 12 makes the Q-value high.

The piezoelectric thin film resonator is used as the acoustic wave resonator 11. As illustrated in FIG. 6A and FIG. 6B, in the piezoelectric thin film resonator, the lower electrode 41 (a first electrode) and the upper electrode 43 (a second electrode) sandwich at least a part of the piezoelectric film 42. The sensitive membrane 45, which is the sensitive part, is located on the opposite side of the upper electrode 43 from the piezoelectric film 42. In the piezoelectric thin film resonator, the resonant frequency and the antiresonant frequency change sensitively to a change in mass of the sensitive membrane 45. Therefore, the detection sensitivity of the sensor circuit is improved.

The antiresonant frequency changes more than the resonant frequency in accordance with the mass change of the sensitive membrane 45. Thus, to improve the detection sensitivity, the acoustic wave resonator 11 is preferably shunt-connected to a signal pathway as illustrated in FIG. 7.

In the resonator 12, the variable capacitor VC1 is connected in parallel to or in series with the acoustic wave resonator 11. This structure enables to adjust the resonant frequency or the antiresonant frequency by adjusting the variable capacitor VC1. Therefore, the oscillation frequency of the oscillation circuit 10 can be adjusted to the frequency at which the sensitivity of the phase shift circuit 18 is high.

### Example of the Phase Shifter 20

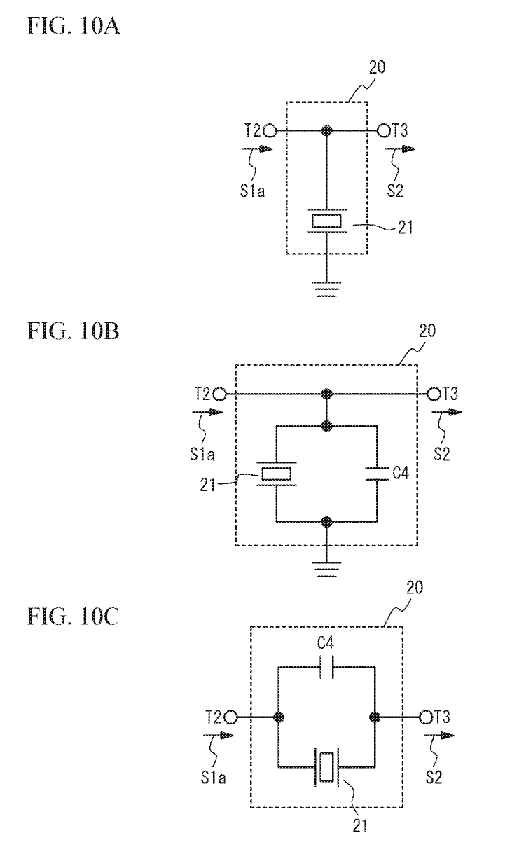

FIG. 10A through FIG. 10C are circuit diagrams of examples of the phase shifter in the first embodiment. In the phase shifter 20 in FIG. 10A, the acoustic wave resonator 21 is shunt-connected between a terminal T2, to which the signal S1a is input, and a terminal T3, from which the signal S2 is output. In the phase shifter 20 in FIG. 10B, the acoustic wave resonator 21 and a capacitor C4 are shunt-connected between the terminals T2 and T3. In the phase shifter 20 in FIG. 10C, the acoustic wave resonator 21 and the capacitor C4 are connected in parallel between the terminals T2 and T3.

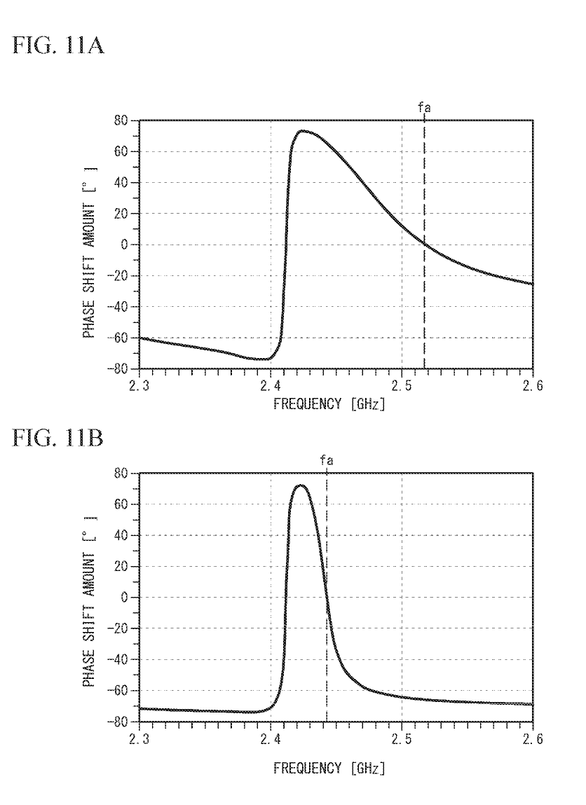

FIG. 11A and FIG. 11B illustrate the phase shift amount with respect to frequency in the phase shifters illustrated in FIG. 10A and FIG. 10B, respectively. As illustrated in FIG. 11A, in the phase shifter 20 in FIG. 10A, the slope of the phase shift amount with respect to frequency is gentle around the antiresonant frequency fa of the acoustic wave resonator 21. Thus, the detection sensitivity to the frequency shift is low.

As illustrated in FIG. 11B, in the phase shifter 20 illustrated in FIG. 10B, the antiresonant frequency fa shifts to a frequency lower than that in FIG. 10A by the capacitor C4. Thus, the slope of the phase shift amount with respect to frequency is steep around the antiresonant frequency fa. Thus, the detection sensitivity to the frequency shift is high.

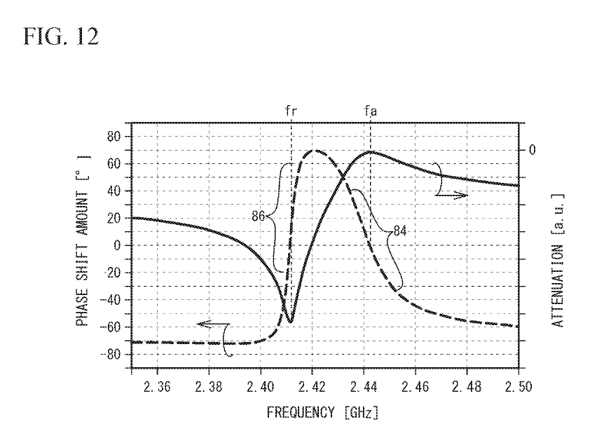

FIG. 12 is a graph of the transmission characteristic and the phase shift amount of the phase shifter in FIG. 10B. The transmission characteristic of the phase shifter 20 is the transmission characteristic from the terminal T2 to the terminal T3. As illustrated in FIG. 12, the attenuation of the phase shifter 20 is large at the resonant frequency fr, and the attenuation is small at the antiresonant frequency fa. As illustrated in FIG. 10A and FIG. 10B, when the acoustic wave resonator 21 is shunt-connected, the attenuation is small in a range 84 around the antiresonant frequency fa. Thus, the insertion loss of the phase shifter 20 is reduced. In addition, the phase shift amount with respect to frequency relatively linearly changes. On the other hand, in a range 86 around the resonant frequency fr, the attenuation is large, and the insertion loss of the phase shifter 20 is thus large. Additionally, the phase shift amount with respect to frequency rapidly changes. Thus, the phase is preferably shifted in the range 84 around the antiresonant frequency fa.

In the phase shifter 20 in FIG. 10C, the attenuation is small around the resonant frequency fr. However, around the resonant frequency fr, the attenuation with respect to frequency rapidly changes. Thus, the frequency dependence of the insertion loss of the phase shifter 20 is large. However, around the resonant frequency fr, a phase shift characteristic is steeper than that around the antiresonant frequency fa. Therefore, the phase shifters 20 in FIG. 10A and FIG. 10B are more preferable than the phase shifter 20 in FIG. 10C. Example of the phase shifter 22

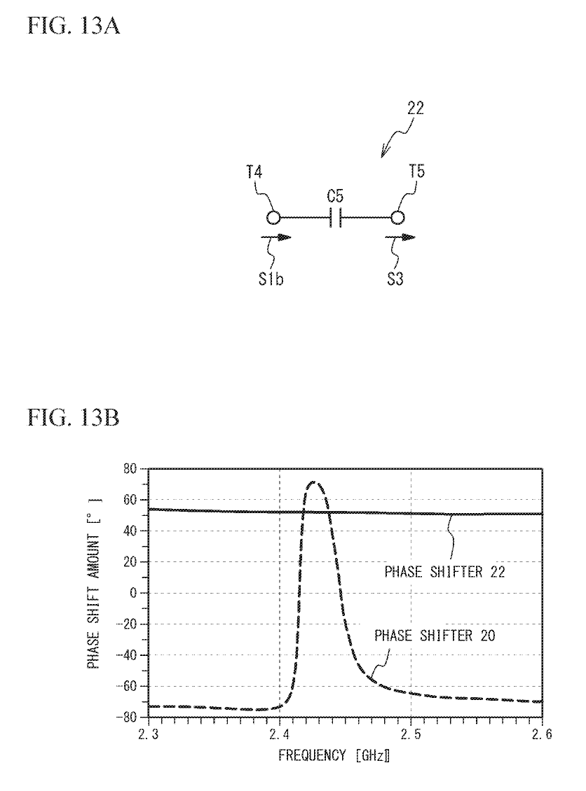

FIG. 13A is a circuit diagram of the phase shifter 22 in the first embodiment, and FIG. 13B illustrates the phase shift amounts of the phase shifters with respect to frequency. As illustrated in FIG. 13A, in the phase shifter 22, a capacitor C5 is connected in series between a terminal T4, to which the signal S1b is input, and a terminal T5, from which the signal S3 is output.

In FIG. 13B, the solid line indicates the phase shift amount of the phase shifter 22, while the dashed line indicates the phase shift amount of the phase shifter 20. The change in the phase shift amount with respect to frequency is small in the phase shifter 22 illustrated in FIG. 13A. Additionally, the phase shift amount is positive. Thus, the phase difference from the phase shifter 20 can be made to be large.

As illustrated in FIG. 10A through FIG. 10C, the phase shifter 20 has the acoustic wave resonator 21 (a second acoustic wave resonator). Thus, the phase shift amount can be greatly changed with respect to a change in frequency of the signal S1a. Therefore, the detection sensitivity of the sensor circuit is improved.

As illustrated in FIG. 10A and FIG. 10B, the acoustic wave resonator 21 is shunt-connected to the transmission line through which the signal S1a is transmitted. Thus, as illustrated in FIG. 12, the insertion loss of the phase shifter 20 is reduced and the frequency dependence of the phase shift amount is made to be nearly linear.

As illustrated in FIG. 10B, the phase shifter 20 includes the capacitor C4, which is connected in parallel to the acoustic wave resonator 21 and shunt-connected to the transmission line. This structure improves the detection sensitivity of the sensor circuit as illustrated in FIG. 11B.

As illustrated in FIG. 12, the frequency of the signal S1a is preferably located at a frequency around the antiresonant frequency fa of the acoustic wave resonator 21. This configuration reduces the insertion loss of the phase shifter 20, and makes the frequency dependence of the phase shift amount nearly linear.

The acoustic wave resonator 21 may be a piezoelectric thin film resonator or a surface acoustic wave resonator. The phase shifter 20 may be other than the acoustic wave resonator 21.

A case where the capacitor C5 is used as the phase shifter 22 is described, but an acoustic wave resonator or the like may be used.

### Second Embodiment

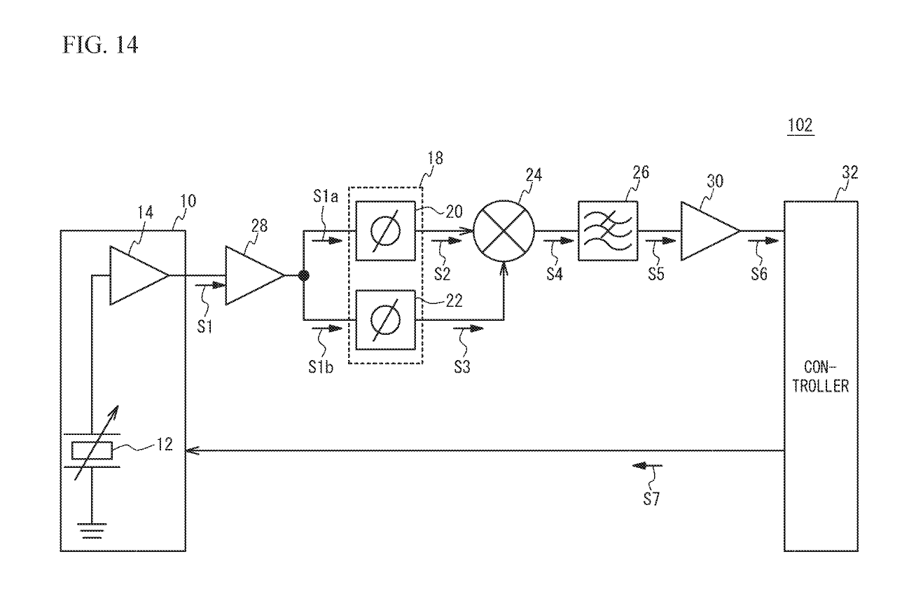

FIG. 14 is a circuit diagram of a sensor circuit in accordance with a second embodiment. As illustrated in FIG. 14, a sensor circuit 102 of the second embodiment differs from the sensor circuit 100 of the first embodiment in that the sensor circuit 102 further includes amplifier circuits 28 and 30 and a controller 32. The amplifier circuit 28 amplifies the oscillation signal S1 of the oscillation circuit 10. The amplifier circuit 30 amplifies the signal S5 output from the LPF 26. An amplified signal S6 is input to the controller 32. The controller 32 is, for example, a processor or a computer, and outputs a signal S7 for adjusting the resonant frequency of the resonator 12 based on the signal S6. Other structures are the same as those of the first embodiment, and the description thereof is thus omitted.

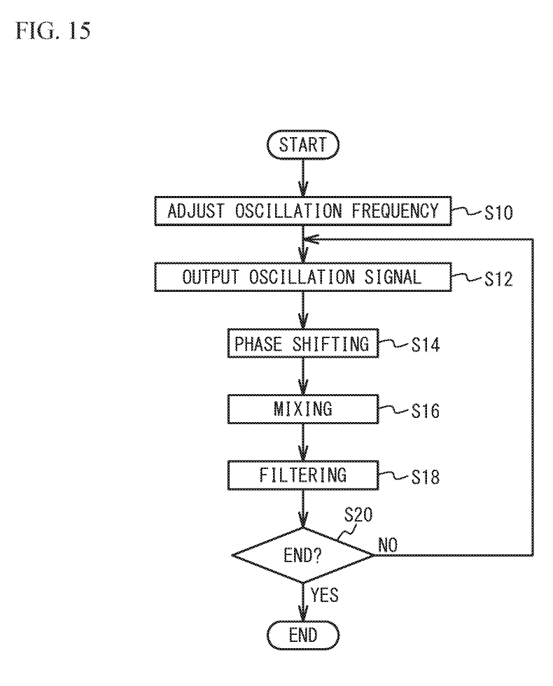

FIG. 15 is a flowchart of a sensing method in the second embodiment. As illustrated in FIG. 12, as an initializing step before the sensor circuit 102 starts sensing operation, the controller 32 adjusts the frequency of the oscillation signal S1 of the oscillation circuit 10 (step S10). For example, the controller 32 outputs the signal S7 to the oscillation circuit 10 so that the frequency of the oscillation signal S1 becomes the reference frequency f0 in FIG. 4. The frequency of the oscillation signal S1 can be adjusted by adjusting the capacitance of the variable capacitor VC1 in FIG. 7 and FIG. 9. For example, the controller 32 feedback-controls the signal S7 so that the signal S6 has a target voltage to adjust the frequency of the oscillation signal S1 to the reference frequency f0. During a sensing period thereafter, the controller 32 fixes the capacitance of the variable capacitor VC1.

When the sensor circuit 102 starts sensing operation, the sensitive membrane 45 is exposed to the environment to be sensed. When the mass of the sensitive membrane 45 changes, the frequency of the oscillation signal S1 of the oscillation circuit 10 changes. The oscillation circuit 10 outputs the oscillation signal S1 of which the frequency has changed (step S12). The amplifier circuit 28 amplifies the oscillation signal S1. The phase shift circuit 18 shifts the phases of the signals S2 and S3 branched from the oscillation signal S1 (step S14). The mixer 24 mixes the signals S2 and S3 (step S16). The LPF 26 filters the mixed signal S4 to extract a low-frequency signal (step S18). The amplifier circuit 30 amplifies the filtered signal S5 and outputs the signal S6 to the controller 32. The controller 32 determines whether to end (step S20). When the controller 32 ends the sensing operation, the determination at step S20 becomes Yes. When the determination at step S20 is Yes, the process ends. When the determination at step S20 is No, the process returns to step S12.

In the second embodiment, as described at step S10 in FIG. 15, the controller 32 adjusts the resonant frequency (the antiresonant frequency) of the resonator 12 prior to the sensing operation. This configuration enables to control the frequency of the oscillation signal S1 to the reference frequency f0 at which the detection sensitivity of the phase shift circuit 18 is good.

The amplifier circuit 28 functions as a buffer amplifier. Accordingly, the frequency of the signal S1 is stabilized. The amplifier circuit 30 amplifies the signal S5. Accordingly, even when the amplitude of the signal S5 is small, the sensor circuit can be operated. Example of the acoustic wave resonator of the resonator

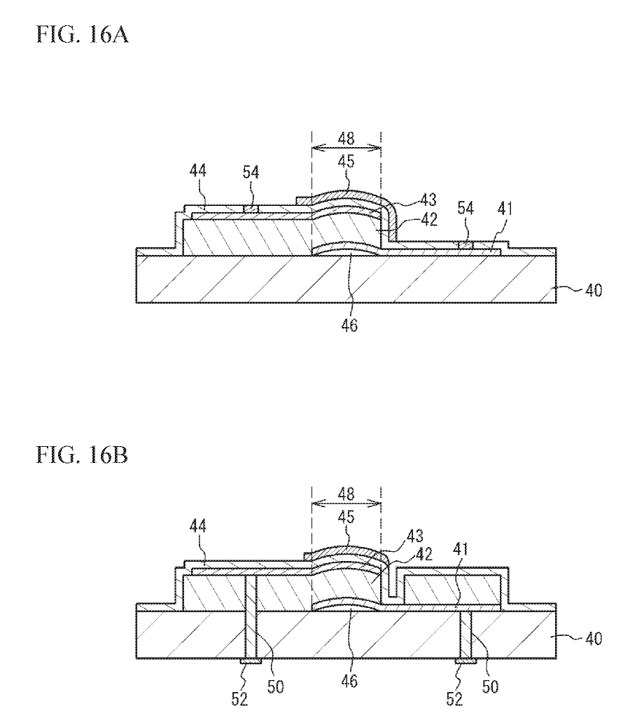

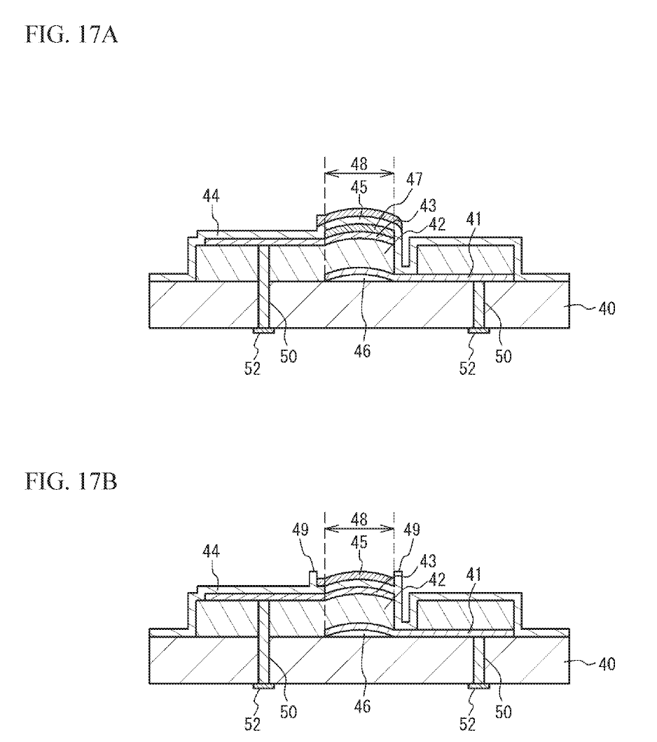

Another example of the acoustic wave resonator 11 of the resonator 12 used in the first and second embodiments will be described. FIG. 16A through FIG. 17B illustrate other examples of the acoustic wave resonator of the resonator in the first and second embodiments. As illustrated in FIG. 16A, instead of providing the through electrodes 50 and electrodes 52, the protective film 44 has apertures, and terminals 54 are located in the aperture. The terminals 54 are electrically connected to the lower electrode 41 and the upper electrode 43. This structure enables to bond a bonding wire to the terminal 54 or conduct a flip-chip mounting with bumps. Other structures are the same as those illustrated in FIG. 6A and FIG. 6B, and the description thereof is thus omitted.

As illustrated in FIG. 16B, the piezoelectric film 42 outside the outer periphery of the resonance region 48 is removed in a groove shape. The Q-value of the acoustic wave resonator 11 is improved by removing the piezoelectric film 42 outside the outer periphery of the resonance region 48. Other structures are the same as those illustrated in FIG. 6A and FIG. 6B, and the description thereof is thus omitted.

As illustrated in FIG. 17A, an additional film 47 for adjusting frequency may be located between the upper electrode 43 and the protective film 44 within the resonance region 48. The resonant frequency can be adjusted by changing the film thickness of the additional film 47. The additional film 47 may be located inside the upper electrode 43, between the piezoelectric film 42 and the upper electrode 43, between the lower electrode 41 and the piezoelectric film 42, or inside the lower electrode 41. Other structures are the same as those of FIG. 16B, and the description thereof is thus omitted.

As illustrated in FIG. 17B, the protective film 44 may have a protruding portion 49 surrounding the resonance region 48. The protruding portion 49 functions as a dam for a solvent in which the material of the sensitive membrane is dissolved when the sensitive membrane 45 is formed on the protective film 44. Other structures are the same as those in FIG. 16B, and the description thereof is thus omitted.

### Examples of the Acoustic Wave Resonator of the Resonator and the Acoustic Wave Resonator of the Phase Shifter

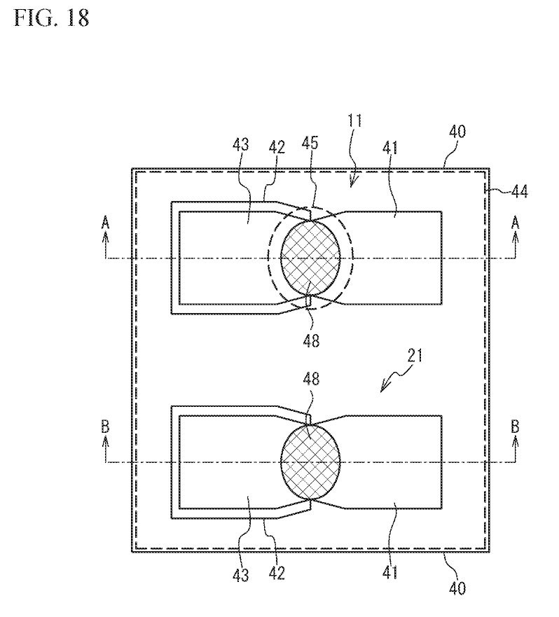

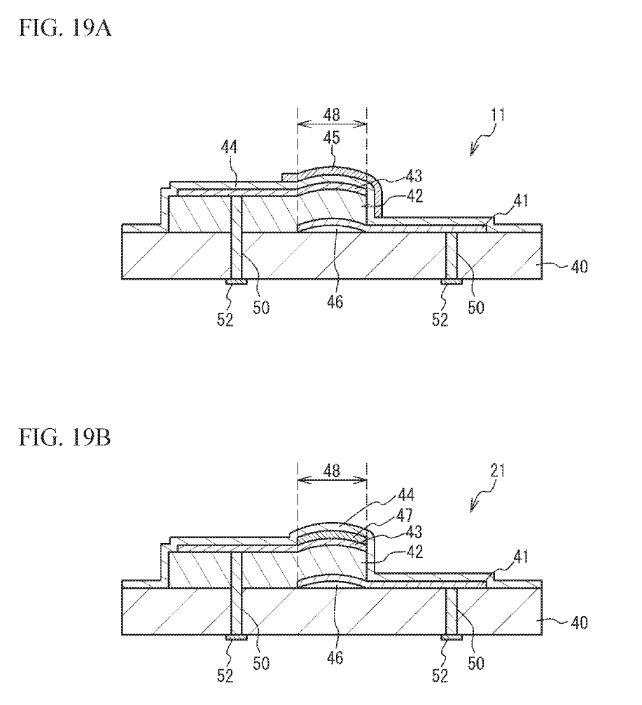

FIG. 18 is a plan view of examples of the acoustic wave resonators of the resonator and the phase shifter in the first and second embodiments. FIG. 19A is a cross-sectional view taken along line A-A in FIG. 18, and FIG. 19B is a cross-sectional view taken along line B-B in FIG. 18. As illustrated in FIG. 18 through FIG. 19B, the acoustic wave resonators 11 and 21 are located on a single substrate 40. The acoustic wave resonator 11 has the sensitive membrane 45 on the protective film 44 within the resonance region 48 but has no additional film 47. The acoustic wave resonator 21 has the additional film 47 between the upper electrode 43 and the protective film 44 within the resonance region 48 but has no sensitive membrane 45. The materials and the film thicknesses of the lower electrode 41, the piezoelectric film 42, and the upper electrode 43 are substantially the same between the acoustic wave resonators 11 and 21. Other structures are the same as those in FIG. 6A and FIG. 6B, and the description thereof is thus omitted.

In FIG. 18 through FIG. 19B, the acoustic wave resonators 11 and 21 are located on the single substrate 40. This structure enables to make the temperatures of the acoustic wave resonators 11 and 21 approximately the same even when the acoustic wave resonator 11 generates heat. In addition, the resonant frequencies (or the antiresonant frequencies) of the acoustic wave resonators 11 and 21 can be adjusted to approximately the same by adjusting the masses of the sensitive membrane 45 and the additional film 47 within the resonance region 48 to approximately the same.

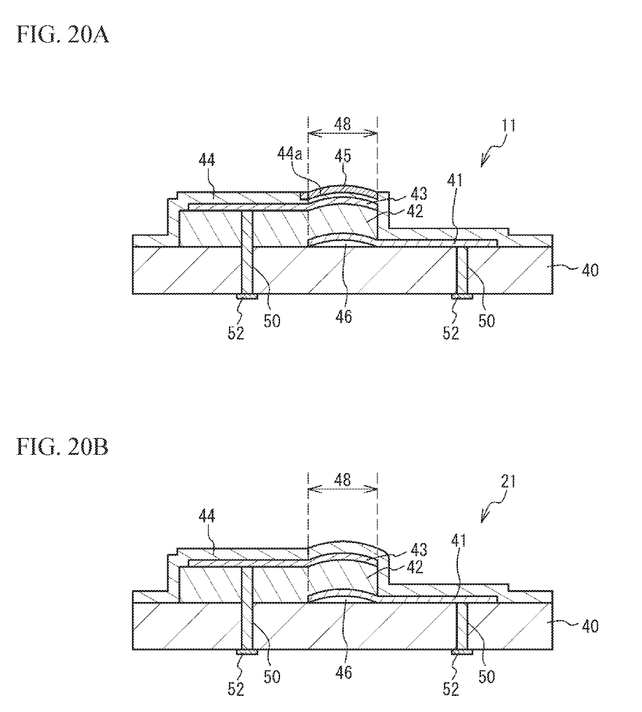

FIG. 20A is another cross-sectional view taken along line A-A in FIG. 18, and FIG. 20B is another cross-sectional view taken along line B-B in FIG. 18. As illustrated in FIG. 20A and FIG. 20B, in the acoustic wave resonator 11, the protective film 44 has a recessed portion 44a. The sensitive membrane 45 is located in the recessed portion 44a. The recessed portion 44a functions as a dam for a solvent in which the material of the sensitive membrane is dissolved when the sensitive membrane 45 is formed on the protective film 44. The acoustic wave resonator 21 has neither the recessed portion 44a nor the sensitive membrane 45. The total mass of the protective film 44 and the sensitive membrane 45 within the resonance region 48 of the acoustic wave resonator 11 is adjusted to be approximately equal to the mass of the protective film 44 within the resonance region 48 of the acoustic wave resonator 21. This configuration enables to adjust the resonant frequencies (or the antiresonant frequencies) of the acoustic wave resonators 11 and 21 to be approximately the same.

The resonant frequency (or the antiresonant frequency) of the acoustic wave resonator 11 can be adjusted with the variable capacitor VC1 or the like. However, the adjustable range of the resonant frequency (or the antiresonant frequency) is limited. Thus, as illustrated in FIG. 18 through FIG. 20B, the resonant frequencies (or the antiresonant frequencies) of the acoustic wave resonators 11 and 21 are preferably adjusted to be approximately the same at the time of manufacturing the acoustic wave resonators 11 and 21.

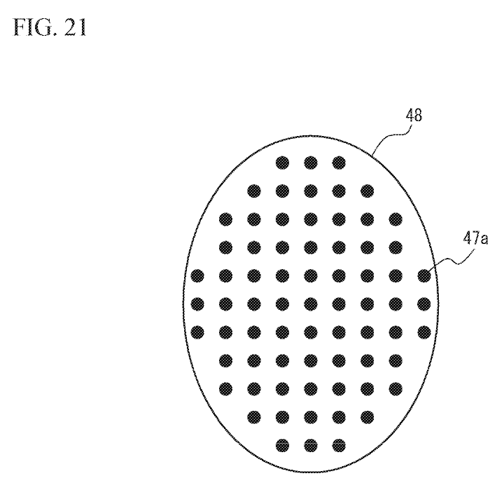

FIG. 21 is a plan view of an additional film in the first and second embodiments. As illustrated in FIG. 21, the additional film 47 within the resonance region 48 may be formed so as to form island patterns 47a. Alternatively, the additional film 47 within the resonance region 48 may have a plurality of apertures. These structures enable to set the resonant frequencies (or the antiresonant frequencies) of the acoustic wave resonators 11 and 21 to desired frequencies.

### Implementation

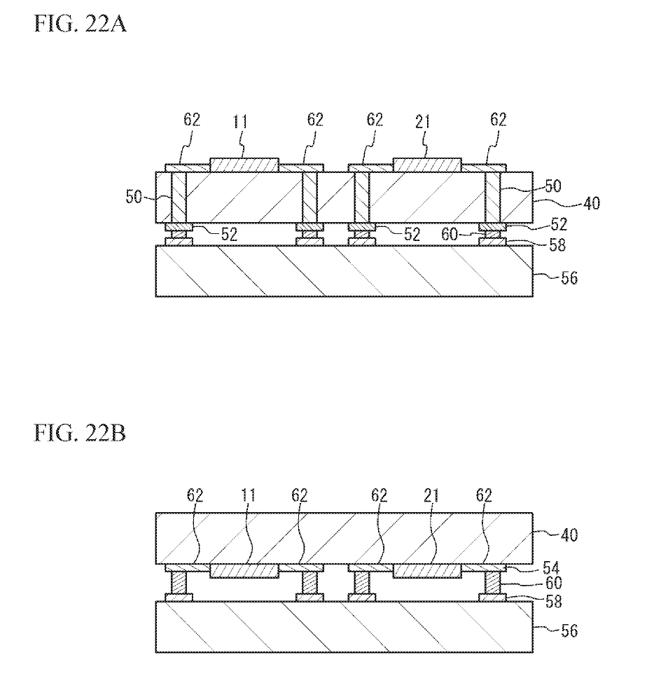

FIG. 22A and FIG. 22B are cross-sectional views of the sensor circuits in the first and second embodiments, respectively. As illustrated in FIG. 22A, the acoustic wave resonators 11 and 21 and wiring lines 62 are located on the upper surface of the substrate 40. The wiring lines 62 are coupled to the lower electrode 41 and the upper electrode 43 of each of the acoustic wave resonators 11 and 21. The electrodes 52 are located on the lower surface of the substrate 40. The through electrode 50 electrically connects the wiring line 62 to the electrode 52. A substrate 56 is a semiconductor substrate such as, for example, a silicon substrate. Circuit elements other than the acoustic wave resonators 11 and 21 are located on the substrate 56. Electrodes 58 are located on the upper surface of the substrate 56. The substrate 40 is face-up mounted on the substrate 56. The electrodes 58 and 52 are bonded together by bumps 60. Other structures are the same as those in FIG. 18 through FIG. 20B.

As illustrated in FIG. 22B, the acoustic wave resonators 11 and 21 and the wiring lines 62 are located on the lower surface of the substrate 40. The substrate 40 is flip-chip mounted on the substrate 56 with use of the bumps 60. Other structures are the same as those in FIG. 22A, and the description thereof is thus omitted.

As illustrated in FIG. 22A and FIG. 22B, the substrate 40 on which the acoustic wave resonators 11 and 21 are formed is mounted on the semiconductor substrate on which circuit elements are formed. This structure reduces the size of the sensor circuit.

Although the embodiments of the present invention have been described in detail, it is to be understood that the various change, substitutions, and alterations could be made hereto without departing from the spirit and scope of the invention.

What is claimed is:

1. A sensor circuit comprising:
a resonator of which a resonant frequency and/or an antiresonant frequency changes as a mass of a sensitive part of the resonator changes;
an amplifier outputting an oscillation signal having a frequency corresponding to the resonant frequency or the antiresonant frequency;
a phase shift circuit changing a phase difference between a first signal and a second signal branched from the oscillation signal in accordance with a change in frequency of the oscillation signal; and
a mixer outputting a signal corresponding to a change in the resonant frequency or the antiresonant frequency of the resonator by mixing the first signal and the second signal between which the phase difference has been changed by the phase shift circuit.

2. The sensor circuit according to claim 1, wherein
the phase shift circuit includes:
a first phase shifter changing a phase of the first signal by a first phase shift amount; and
a second phase shifter changing a phase of the second signal by a second phase shift amount,
wherein an amount of change in the second phase shift amount with respect to a change in frequency of the second signal differs from an amount of change in the first phase shift amount with respect to a change in frequency of the first signal.

3. The sensor circuit according to claim 2, wherein
the first phase shifter includes a first acoustic wave resonator.

4. The sensor circuit according to claim 3, wherein
the first acoustic wave resonator is shunt-connected to a transmission line through which the first signal is transmitted.

5. The sensor circuit according to claim 4, wherein
the first phase shifter is connected in parallel to the first acoustic wave resonator and is shunt-connected to the transmission line.

6. The sensor circuit according to claim 4, wherein
the frequency of the first signal is located around an antiresonant frequency of the first acoustic wave resonator.

7. The sensor circuit according to claim 1, wherein
the resonator includes a second acoustic wave resonator.

8. The sensor circuit according to claim 7, wherein
the second acoustic wave resonator includes:
a piezoelectric layer;
a first electrode and a second electrode sandwiching at least a part of the piezoelectric layer; and
a sensitive membrane that is located on an opposite side of the second electrode from the piezoelectric layer and is the sensitive part.

9. The sensor circuit according to claim 1, further comprising:
a low-pass filter coupled to an output terminal of the mixer and having a cutoff frequency lower than the frequency of the oscillation signal.

10. The sensor circuit according to claim 1, further comprising:
a controller adjusting the resonant frequency and/or the antiresonant frequency of the resonator prior to sensing operation.

11. A sensing method comprising:
outputting an oscillation signal having a frequency corresponding to a resonant frequency or an antiresonant frequency of a resonator, the resonant frequency or the antiresonant frequency changing as a mass of a sensitive part of the resonator changes;
changing a phase difference between a first signal and a second signal branched from the oscillation signal in accordance with a change in frequency of the oscillation signal; and
outputting a signal corresponding to a change in the resonant frequency or the antiresonant frequency of the resonator by mixing the first signal and the second signal between which the phase difference has been changed.
